(19) United States
(12) Patent Application Publication
(10) Pub. No.: US 2023/0173220 A1
(43) Pub. Date: Jun. 8, 2023

(54) AROMA TRAINING USING AUGMENTED REALITY

(71) Applicant: Accenture Global Solutions Limited, Dublin (IE)

(72) Inventors: Marc Carrel-Billiard, Vence (FR); Alex M. Kass, Palo Alto, CA (US); Derrick Anthony Martin, II, Brooklyn, NY (US); Alexandria Emily Pabst, Merced, CA (US)

(21) Appl. No.: 17/875,080

(22) Filed: Jul. 27, 2022

Related U.S. Application Data
(60) Provisional application No. 63/286,744, filed on Dec. 7, 2021.

(51) Int. Cl. A61M 21/00; G16H 20/70

(52) U.S. Cl. CPC A61M 21/00; G16H 20/70; A61M 2021/0016; A61M 2021/005; A61M 2230/10

(57) ABSTRACT

Methods, systems, and apparatus, including computer programs encoded on a computer storage medium, for implementing an olfaction training program by an online service executing on a server using an AR enabled headset. The objects in the vicinity of the users are identified and highlighted. If the objects have an aroma, the objects are used for olfaction tests. The user interacts with the AR environment to select an object in response to detecting an aroma. The user responses is transmitted back to the online service. The online service determines a subsequent object based on the user response. The subsequent object is then highlighted in the AR environment and the user is prompted to take a subsequent olfaction test.

### CROSS-REFERENCE TO RELATED APPLICATION

This application claims the benefit of U.S. Provisional Application No. 63/286,744, filed Dec. 7, 2021, and titled “Aroma Synthesis for Olfactory Training,” which is incorporated by reference.

### TECHNICAL FIELD

This specification relates to olfactory based multisensory augmented reality, to generate a sequence of stimuli for users based on user activity.

### BACKGROUND

Olfaction is one of the five primary human senses that allows humans to experience the physical world. It plays an important role in detecting hazards such as fire or toxic fumes while also allowing humans to enjoy food. Olfaction deficiency (OD) can therefore be associated to a plurality of health issues such as depression, decreased nutrition which can have a negative impact on the quality of life. Due to the COVID-19 pandemic, there is an increase in the number of people suffering from post-viral olfactory dysfunction (PVOD) resulting in a large number of people to have OD with no promising cure.

Augmented reality (AR) refers to an interactive experience of a physical real-world environment where components of the physical real-word environment are augmented using digital information that allows for an enhanced perception of the physical real-world using visual, auditory, haptic, somatosensory and olfactory senses. The user can interact with the augmented environment via electronic hardware such as an AR headset with a display screen, gloves or other clothing/accessories that are fitted with sensors (such as motion sensors), actuators, etc. Recent advances in technology has led AR technology to expand beyond the audio-visual and virtual motion setting by incorporating the sense of aroma (olfaction). This is generally achieved by adding a device such as an atomizer that can include a dispersing agent and an electronically controlled valve to an AR headset that can be used to release dispersing agent to generate aroma conditions in a 3D virtual environment. The AR headset can also include a sensor that can mimic human olfaction that can detect aromas present in the physical real-world.

### SUMMARY

This specification generally describes a techniques and methods of generating OD treatment plan in an AR environment using an AR headset capable of dispersing aroma. The techniques and methods are implemented in a distributed environment where users undertake an OD treatment using AR headsets. The techniques described in this document can allow a user suffering from OD undertake a treatment plan that is made interesting to the user by designing and planning the treatment as a fun exercise. For example, the physical environment around the user can be augmented and presented to the user as a game where the user has to go through multiple stages where during each stage of the game the user can complete an olfaction task to score some points. In some situations, the game can also be presented as a multi-user game where more than one user can participate and the users can collectively or independently try to score points as a competition.

The techniques and methods described in this specification can be used in different variations to as to customize the treatment as per the likings of each individual user thereby making more and more users undertake the treatment plan. For example, some users may prefer the treatment as a game that involves storytelling however other user may want the treatment as an examination where the users can score points based on their responses.

The techniques further include gathering AR experiences of multiple users and using those experiences to further enhance the treatment plan. For example, based on AR experiences that includes olfaction test results of multiple users, patterns can be identified that can be used to generate and update treatment protocol over time. For example, if majority of the users are able to perceive an aroma at a particular intensity at a particular distance from an object, it is highly likely that a new user will also be able to perceive the aroma in the same conditions. If a user fails to detect the aroma it would indicate how the user's OD conditions differentiates from the rest of the users which might require further assistance during the treatment plan.

In general, one innovative aspect of the subject matter described in this specification can be embodied in methods including the operations of receiving from a client device of an user, visual data indicating a plurality of objects in the vicinity of the client device and aroma data indicating the aroma of each of the plurality of objects; transmitting to the client device, instructions to present a first set of visual indicators to the user based on the visual data and the aroma data; receiving from the client device, a user response that was provided by the user in response to the presentation of the first set of visual indicators; storing data in a database wherein the data includes the user response, the first set of visual indicators of the plurality of objects and the aroma data; generating a data model using the data stored in the database wherein the data model is configured to generate as output, a second set of visual indicators for presentation to the user of the client device; transmitting, to the client device, the second set of visual indicators for presentation to the user of the client device.

Methods can include the visual data to include data indicating the plurality of objects in a three dimensional environment of the user of the client device collected using a camera of the client device.

Methods can include aroma data to include data indicating the aroma of one or more objects in the plurality of objects collected using an aroma sensor of the client device.

Methods can include the first set of visual indicators to include (i) visual data identifying one or more objects from among the plurality of objects, (ii) visual data depicting the intensity of aroma of the one or more objects.

Methods can include instructions to highlight the first set of visual indicators to identify the one or more objects from among the plurality of objects.

Methods can include the user response provided by the user in response to the presentation of the first set of visual indicators to include (1) an indication whether the user was able to perceive the aroma of the one or more objects, (2) an indication whether the user is able to associate the aroma of the one or more objects with the one or more objects displayed on the client device, and (3) a score provided by the user indicating the level of confidence the user has on the association of the one or more aromas of the one or more objects to the one or more objects present by the client device.

Methods can include the user response provided by the user to further include electrical activity of the brain of the user collected using one or more electrodes of the client device affixed to the scalp of the user wherein the electrical activity of the brain identifies brain activity in response to the user smelling the first object.

Methods can further include identifying one or more users from among the plurality of users based on prior user responses; generating a training dataset based on data stored in the database of the one or more users; generating the data model based on the training dataset that further comprises: generating a rule-based data model that includes a set of rules that generates as output, the second data that indicates the second object and the second smell; generating a machine learning data model that is trained on the one or more identified training datasets to generate as output, the second data that indicates the second object and the second smell.

Particular embodiments of the subject matter described in this specification can be implemented so as to realize one or more of the following advantages. The techniques and methods described in this specification generates data points indicating user responses to different types of aroma. Patterns in such data is used to create a treatment plan for users suffering from OD. The techniques allow the users to participate in the treatment from either home or any location preferred by the user. The techniques further allow to change the treatment plan according to the user requirements so that the users can be provided with a custom user-specific treatment plan that will result in a faster recovery from OD. The treatment plan can be further customized or changed while the treatment is ongoing.

The techniques and methods implements state-of-the-art machine learning models and rule based model to learn the intricate relationships in the data that would generally go unnoticed. Use of such models to treat OD results in a faster recovery of users from OD. The techniques and methods are further implemented in a way that allows distribution of processing between servers implementing the treatment plan and AR headsets so as not bottleneck either one of them. The methods also allow generating of treatment plans ahead of time and downloaded on the AR headset so as to not use valuable computing resources when the user is undertaking the test or when the user is not connected to the internet.

The details of one or more embodiments of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example system that implements an online treatment service for users suffering from olfaction deficiency.

FIG. 2 is a block diagram that illustrates two strategies of presenting objects in virtual reality.

FIG. 3 is a block diagram illustrating an example OTS.

FIG. 4 is a flow diagram of an example process of the olfaction treatment service.

FIG. 5 is a block diagram of an example computer system.

### DETAILED DESCRIPTION

This specification generally describes a techniques and methods of generating OD treatment plan in an AR environment using an AR headset capable of detecting and dispersing aroma. The techniques and methods are implemented in a distributed environment where users undertake an OD treatment using AR headsets. It should be noted that the techniques and methods described in this document can also find applications in other areas such as gaming, education and engineering using virtual reality. For example, in the field of education, a teaching plan can be generated using the techniques and methods described here that allows for a better learning experience.

The techniques and methods described in this specification can be used in different variations to as to customize the treatment as per the likings of each individual user thereby making more and more users undertake the treatment plan. For example, some users may prefer the treatment as a game that involves storytelling however other user may want the treatment as an examination where the users can score points based on their responses. As an example, the user of the AR headset who prefers the treatment as a game. These techniques can allow the treatment plan to include more comprehensive gaming styles such as the user enacting a role of a detective who is trying to solve a mystery using the sense of smell. The techniques also allow the AR headset to record brain activity of the user in response to the user smelling an aroma. To enhance the training for each user by integrating electroencephalogram (EEG) and AR into the headset would to provide a greater degree of sensory immersion for the user of the AR headset

The techniques further include gathering AR experiences of multiple users and using those experiences to further enhance the treatment plan. For example, based on AR experiences that includes olfaction test results of multiple users, patterns can be identified that can be used to generate and update treatment protocol over time. For example, if majority of the users are able to perceive an aroma at a particular intensity at a particular distance from an object, it is highly likely that a new user will also be able to perceive the aroma in the same conditions. If a user fails to detect the aroma it would indicate how the user's OD conditions differentiates from the rest of the users which might require further assistance during the treatment plan.

The techniques further allow using the AR experiences to generate machine learning models to learn intricate relationships that may otherwise be undetected. The machine learning models can be further used to create a treatment plan that will prove to be more beneficial to the user. For example, the machine learning model can generate a sequence of aromas with different intensities that can result in the user identify aromas easily and at a faster rate thereby giving the users a sense of recovery.

It should be noted that the techniques and methods described in this document can also find applications in other areas such as gaming, education and engineering using virtual reality. For example, in the field of education, a teaching plan can be generated using the techniques and methods described here that allows for a better learning experience.

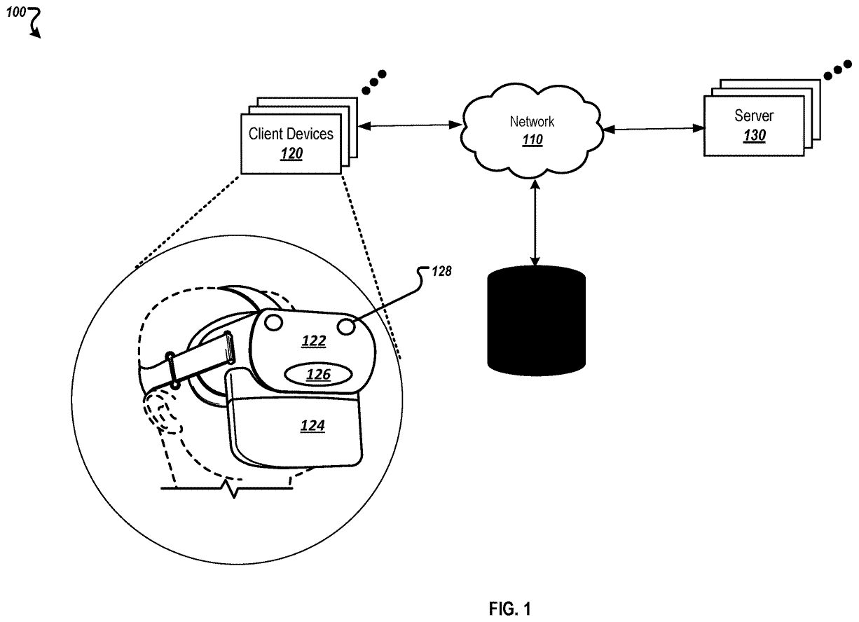

FIG. 1 is a block diagram of an example distributed environment 100 that can implement an OD treatment program. The environment 100 includes a network 110. The network 110 can include a local area network (LAN), a wide area network (WAN), the Internet or a combination thereof. The network 110 can also include any type of wired and/or wireless network, satellite networks, cable networks, Wi-Fi networks, mobile communications networks (e.g., 3G, 4G, and so forth) or any combination thereof. The network 104 can utilize communications protocols, including packet-based and/or datagram-based protocols such as internet protocol (IP), transmission control protocol (TCP), user datagram protocol (UDP), or other types of protocols. The network 104 can further include a number of devices that facilitate network communications and/or form a hardware basis for the networks, such as switches, routers, gateways, access points, firewalls, base stations, repeaters or a combination thereof. The network 110 connect client devices 120 such as AR headsets and one or more server systems 130.

Client devices 120 such as AR headsets are generally heads-up display (HUD) that allows users to interact with an AR which is an augmented real-world environment in a first-person view (FPV). The AR headset can display digital content in the AR environment. As used throughout this document, the phrases “content” refer to a discrete unit of digital content or digital information (e.g., a video clip, audio clip, multimedia clip, image, text, or another unit of content). Content can be electronically stored in a physical memory device as a single file or in a collection of files, and content can take the form of video files, audio files, multimedia files, image files, or text files and include advertising information. Audio content may be presented by external devices (e.g., speakers) and/or devices that are internal to the AR headset (e.g., headphones).

AR headsets typically include one or more cameras to perceive the real-world environment around the user of the AR headset, an electronic display 122, a rendering apparatus that uses the electronic display 122 to render the real-world environment recorded by the camera 128 and digital content that augments the real-world environment, an array of sensors (e.g., motion tracking sensors, head and eye tracking sensors and positional sensors) that keep track of the user motion and alignment with respect to the AR environment and the physical environment around the user. AR headsets are capable of communicating with other entities over the network 110. For example, AR headsets are capable of requesting, receiving and transmitting digital content over the network 110. AR headsets can also include an electronically controlled aroma dispenser 124 which is an atomizer sprayer that can spray one or more different perfumes that simulate one or more different types of aromas at different intensities. AR headsets can also include an aroma sensor 126 that can detect different types of aroma present in the real-world environment. For example, the aroma sensor 126 can detect different types of aroma within the vicinity of the user of the AR headset.

In some implementations, the AR headset can also include one or more electrodes that can record brain activity of the user in response to the user smelling an aroma. The electrodes for example, can be attached to the scalp of the user using the AR headset. These electrodes can be inbuilt into the AR headset or they can be a separate attachment that can be installed on the AR headset using an interface.

The AR headset 120 typically includes a user application, such as AR based gaming applications and/or AR based applications provided by health care providers that can facilitate the sending and receiving of digital content and instructions over the network 110. The user application 126 is also the application that can use the components of the AR headset to present digital content to the user of the AR headset. The digital content can include visual data such as images or a stream of images that forms the real-world environment recorded using the one or more cameras 128 and visual indicators that augments different components of the real-world environment. For example, the electronic display 122 can display an AR environment depicting a room in where the user of the AR headset is present where one or more objects present is the room is highlighted using visual indicators.

In some implementations, if the user of the AR headset is suffering from OD and wants to engage into a series of tests to check the stage of OD that the user is suffering from, the user can select an application provided by a healthcare provider. The application can communicate with one or more servers that provide an online OD testing service. The application can exchange data over the network 110 to present digital content depicting the augmented real-world environment to the user of the AR headset. The AR headset also disperses aroma that is associated to the one or more objects at different intensities to check if the user's olfactory sense is able to pick up the aroma.

For example, if the user of the AR headset walks into a kitchen, the camera 128 of the AR headset can record the real-world environment of the kitchen. Simultaneously, the aroma sensor 126 can also detect different aromas present in the kitchen. The AR headset 120 then transmits the recorded visual data and aroma data to the one or more servers that provide an online OD testing service. The OD testing service can process the recorded visual data and aroma data to identify one or more objects present in the real-world environment and assigns the different aromas to the one or more objects. The aromas are assigned to the objects based on the objects characteristics. For example, if the identified object is an “orange”, and the aroma data represents a “citric” aroma, the OD testing service can determine a positive correlation between the “orange” and the “citric” aroma.

The OD testing service in response to receiving and processing the visual data and aroma data, transmits a first set of visual instructions to the AR headset that when executed on the AR headset, highlights the identified object while presenting the real-world environment. For example, the AR headset can highlight the “orange” if the OTS decides to test if the user of the AR headset is able to perceive the aroma of the orange. In some implementations, if the user is not able to perceive the aroma, the AR headset can increase the intensity of the aroma by dispersing aroma using the aroma dispenser 124. The iterative process can thereby allow the OD testing service to determine the severity of the OD.

As for another example, if the user of the AR headset is suffering from OD wants to undertake a treatment program to cure the OD, the user can select another application provided by a health care provider. The application can communicate with one or more servers 130 that provide an olfaction treatment program as an online service (referred to as an Online Treatment Service (OTS)) to allow the user to recognize aromas that the user was capable of before suffering from OD. The application can receive instructions to release dispersing agent associated to an object present in the vicinity of the user that was recorded and identified by the AR headset. After receiving the instructions, the application highlights the object by displaying the digital content in the AR environment and executes instructions that results in releasing the dispersing agent. For example, the AR headset can highlight an object such as an “apple” and increase the intensity of the aroma of the “apple”. Similarly, the AR headset can highlight a cup of coffee and the instructions can cause release of dispersing agent that has a coffee like aroma.

The user of the AR headset can interact with the AR environment to indicate whether the user is able to perceive an aroma of the highlighted object, and whether the user is able to conclude that the aroma is related to the object highlighted in the AR environment. The AR headset transmits the user interaction to the OTS and the OTS can transmit instructions to present the next best alternative object and an associative aroma to the user. The OTS can determine the next best alternative object and the associated aroma based on historical data including user interactions and data collected from other users. The following description provides an in-depth view of the techniques and methods of implementing a treatment program.

The OTS is an online service that can be provided by a health care provider to manage and generate a treatment program for the users using servers 130. For example, the OTS can manage user profiles of the users enrolled into the treatment program including recording and updating data regarding ongoing treatment program of each user. For example, the OTS is responsible for managing the database 150 that stores user profiles, creating a training program for all users, customizing training program for one or more users etc. The OTS for example is also responsible for communicating with AR headsets of the users and transmit information such as digital content, data and instructions required to execute the treatment plan on the client side.

In some implementations, new users can join the olfaction training program provided by the OTS by registering with the provider of the OTS. The registration can include filling out an online form provided by the provider. For example, the new users can use a browser based application from a client device such as a PC, tablet or a smartphone to navigate to a webpage that includes the registration form via a universal resource locator (URL). After being presented with the registration form, the users can provide one or more details according to the registration form. For example, the users can provide their name, gender, email, mobile number etc. In some implementations, users can also provide details of their healthcare insurance one or more details regarding prior or current health conditions including basic physical attributes such as weight, height and age. Upon submission of the details, the OTS can generate a profile for the user and store it in a database 150. In some implementations, the OTS can add one or more other details to the user profile that for example can identify the user as a new user which can be leveraged by the service provider to teach and decide a training program best suited for beginners.

In some implementations, each user profile can also include user status data that can incorporate information such as log data indicating the training sessions that the user has undertaken. For example, the log data for a particular user can include times stamps of training sessions that the particular user of the AR headset took since the time the particular user enrolled into the olfaction training program. Log data can include information such as the software and hardware configuration of the AR headset including the application that is being executed in the AR headset, the geographical location of the AR headset and the quality of the network to which the AR headset is connected to.

In some implementations, user status data can also include historical data indicating different objects and the associated aromas that the user came across in the AR environment. For example, an instance of the log data includes the time stamps of a training session undertaken by a particular user. Historical data can also include (or point to) the digital content that was presented to the user, the object of the AR environment, data identifying the associated aroma of the object, the intensity of the aroma of the object (recorded using the aroma sensor) and whether or not the OTS dispersed aroma to increase the intensity of the aroma.

In some implementations, user status data can also include user responses that were recorded by the AR headset and transmitted to the OTS. The user responses can sometimes be for example, user interactions that indicate can outcome of a positive or a negative identification of an aroma of an object depicted in the AR environment, a score that indicates an association between the object and the aroma or brain activity recorded by the electrodes of the AR headset. For example, a user of an AR headset can be presented with multiple highlighted objects. The user can then be asked to identify the object based on a particular aroma detected by the aroma sensor 126. The user can interact with the AR environment (for example, via hand gestures) to select one of the objects as the user selection. In some implementations the user can provide a score indicating the confidence that the user has while associating the particular aroma to one or more objects.

If the AR headset includes electrodes to record brain activity, then user responses can also include fluctuations in electrical activity of the user's brain. The OTS can use the signals that identify olfaction to determine when aromas are being detected, independent of whether the patient is consciously aware or able indicate detection. It may also be used to detect fluctuations in intensity of the perceived aroma. This can be measured through changes both in activity location, and across time through electroencephalogram (EEG) methods of source localization and traditional signal analysis such as event related potential (ERP) or time-frequency analysis (TF).

In some implementations, the brain activity signals can also be used as additional input to machine learning models (described later) that can classify brain activity to determine the valence of the odor (pleasant or unpleasant) and the kind of odor detected. This can also provide additional data to help the machine learning models develop better precision in less time. In some implementations, the additional data can also be uploaded to data repositories to contribute to studies of population-level effects for improving general smell-disorder treatment protocols. Through measurement of brain signals, the system can adapt aroma training to the individual. For example, even if a user cannot subjectively identify different aromas, their brain may recognize that the odor is present, in response to which more aroma can be dispersed at a higher intensity or for a longer time interval until the user can subjectively detect the odor's presence.

In some implementations, the OTS can generate an OD treatment program that includes one or more sessions. Each session can include one or more olfaction tests where for each of the tests, the user suffering from OD is required smell and identify or correlate objects having similar aroma. In some implementations, the OTS and the application can implement the treatment program as a game to increase user's engagement with the treatment program. For example, the treatment session can include multiple olfaction tests where the user can score points by successfully identifying an object based on the aroma of the object.

In some implementations, the treatment plan can be made interesting by designing and planning the treatment as a fun exercise. For example, the AR environment that is presented to the user can be a designed as a game where the user has to go through multiple stages where during each stage of the game the user can complete an olfaction task to score some points. In some situations, the game can also be presented as a multi-user game where more than one user can participate and the users can collectively or independently try to score points as a competition.

In some implementations, the treatment session can include multiple olfaction tests based on a level of difficulty. For example, in some circumstances it can be assumed that it is easier to identify the aroma of coffee beans than identifying the aroma of some rare fruits. In such implementations, the OTS can select a difficulty level of an olfaction test based on a set of rules or a machine learning model trained based on the data collected by the OTS from all the users and their olfaction tests. For example, a rule based implementation of selecting the level of olfaction test can increment the level of difficulty based on a correct identification of a highlighted object from among multiple highlighted objects based on the aroma of the object in the AR environment.

In some implementations, the OTS can record AR experiences of multiple users and can use experiences to further enhance the treatment plan. For example, based on AR experiences that includes olfaction test results of multiple users, patterns can be identified that can be used to generate and update treatment protocol over time. For example, if majority of the users are able to perceive an aroma at a particular intensity at a particular distance from an object, it is highly likely that a new user will also be able to perceive the aroma in the same conditions. If a user fails to detect the aroma it would indicate how the user's OD conditions differentiates from the rest of the users which might require further assistance during the treatment plan.

In some implementations, when a user initiates a treatment session using the application of the AR headset, the AR headset communicates with the server 130 to notify the OTS to initiate a treatment session. In response, the OTS can access the user profile to analyze historical records of the user and the status of the user's training program to determine an object for a subsequent olfaction test. For this the OTS can implement one or more rule-based models or machine learning models. The OTS can use the rule-based models and the machine learning models to determine based on the historical records, the previous treatment sessions undertaken by the user and how well the user performed in the olfaction tests in the previous sessions.

In some implementations, the OTS can analyze the data to explore patterns in the data collected from the users, the user profiles and test results to determine complex relationships that can be further exploited to improve the user experience and accelerate OD recovery. For example, the server can determine based on prior olfaction testing results, a sequence of olfaction tests that results in an incremental triggering of user memory resulting in user remembering particular type of aromas prior to suffering from OD. The server after determining such patterns and due to a high success rates across multiple users, can create a testing plan for each user based on such patterns

In some implementations, the OTS can determine patterns in the historical data that can be used to determine the olfaction tests of the recently initiated treatment session that the user is going to undertake. For example, assume that a particular user came across citrus fruits multiple times in the AR environment and the particular user failed majority of the times to correctly identify the citrus fruit based on the aroma. In such a situation the OTS can conclude that the particular user needs to improve OD for citrus aroma. Having concluded what kind of aroma needs to be presented to the user, the OTS can select objects that have a citrus aroma for presentation to the user.

As for another example, the OTS can determine based on historical data, a sequence of olfaction tests that includes particular types of digital content representing an object or an environment and an associated aroma that results in an incremental triggering of user memory resulting in user remembering particular type of aromas prior to suffering from OD. The OTS after determining such patterns and due to a high success rates across multiple users, can create a treatment program for each user.

As for another example, the OTS can generate one or more groups of users based on the user profiles and historical data. For example, the OTS can generate groups of users having one or more similar characteristics. For example, the OTS can create a user group that include only those users who have completed a certain number of training sessions. As for another example, the OTS can create a user group that includes users who for example, can correctly identify a particular aroma. The OTS can determine a sequence of olfaction tests based on such user groups. For example, a user in a particular group is likely to make similar mistakes during an olfaction test as other member of the particular group. Hence the user of the particular group should be undertake the same olfaction test based on the assumption that the user is similar to other members of the particular group and taking the test will make the user identify aromas not known the user.

In some implementations, the OTS can also implement one or more machine learning models to model the progression of the user's treatment. The machine learning models implemented by the OTS can include training parameters that can be trained on historical data to predict objects from the multiple objects in the AR environment that when included in the olfaction test for the user, will improve user's OD by progressively making the user identify different aromas that the user was previously familiar with.

In some implementations, the one or more machine learning models implemented by the OTS are configured to receive as input information from the user profile, user status data and historical data. For example, one of the machine learning models can be a neural network model that includes multiple neural network layers and each neural network layer can include multiple training parameters. The neural network can receive as input information from user profile include for example, age, gender and the date from when the user started showing symptoms of OD. The neural network can also receive as input information from one or more previous olfaction tests. For example, the object that the user of the AR headset comes across, the user response etc. The neural network can also be configured to receive user group characteristics as input. The neural network model can process the input and generate as output a prediction indicating one or more objects that are present in the user's vicinity that can be highlighted for the user in the forthcoming olfaction test. Though the above example has been explained using a neural network model, the OTS can implement any machine learning model known in the art. For example, clustering, support vector machines (SVM).

The machine learning models implemented by the OTS can be trained on a training dataset that can be extracted from the user profile and user status data. For example, the training dataset can include training samples of multiple users where the training samples can include features such as gender, age, prior olfaction tests and results, score and electrical brain activity. In some implementations, the training dataset can also be a time-series that includes multiple training samples for multiple users sorted based on the users and the time of the olfaction tests.

Upon determination of the one or more object that can be highlighted to the user for a subsequent olfaction test, the OTS can transmit instructions to present a set of visual indicators to the user based on the visual data highlighting the object and instructions for releasing dispersing agent if necessary. In some implementations, the AR headset after receiving the instructions from the OTS, highlights the object in the AR environment of the user of the AR headset.

In some implementation, the OTS can provide additional instructions to the application of the AR headset to implement different strategies and/or methods for the olfaction tests. The OTS can specify what type of response is expected from the user of the AR headset in response to highlighting one or more objects. For example, the OTS can provide instructions to the application to highlight multiple objects in the user's vicinity and provide the user with an option of selecting any one or the multiple highlighted objects based on the aroma present in the user's environment. The user can see the multiple highlighted objects in the AR environment and guess whether there is any aroma and whether the detected aroma is associated with any of the multiple objects. Depending upon this guess, the user can select an object in the AR headset. For example, the user can use external controllers or an eyeball tracking sensor in the AR headset to select an option. In some implementations, the OTS can also ask the user via the application of the AR headset to provide a score for the user's selection indicating the confidence the user has on the selection. For example, the OTS can instruct the application of the AR headset to present a scale in the AR environment and the user can select a score value from the scale by gazing at the score value on the scale.

In some implementations, the OTS can provide additional instructions to the application to disperse aroma related to one of the objects detected in the AR environment when the aroma sensor 126 fails to detect aroma corresponding to the multiple detected objects or when the intensity of a detected aroma is relatively low for the user with OD to detect.

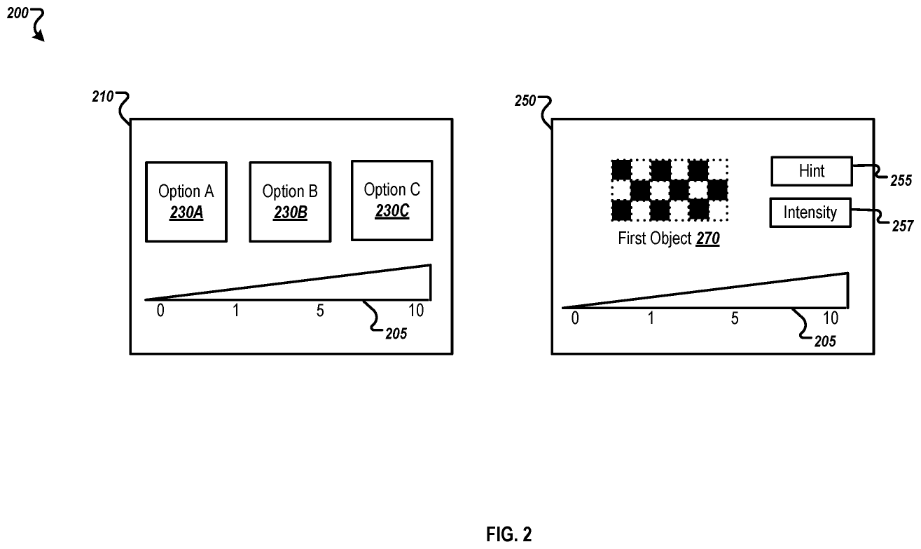

In some implementations, when the AR headset and the OTS detects an aroma of an object present in the AR environment of the user, the OTS can provide instructions to blur the image of the object or hide the object by displaying a pattern overlayed on the object. In such a situation the user can be further instructed to identify the object based on the aroma and provide the name of the object as speech using the microphone of the AR headset. In such a situation, the user can also be provided with an option of hint, wherein the user of the AR headset can reduce the blurriness of the object. By reducing the blurriness of the object and by smelling the aroma, the user of the AR headset can identify the object. As for another example, the OTS can provide additional instructions to the application to increase the intensity of the aroma of the object to help the user guess whether the aroma is associated to the object that is presented by the AR headset. Few of these strategies are explained further with reference to FIG. 2.

FIG. 2 is a block diagram that illustrates two strategies of presenting objects in AR. The two strategies are depicted as 210 and 250. For example, 210 represents a strategy, where the AR headset presents multiple objects 230A-C. The user of the AR headset, after perceiving the aroma, can select one of the objects from the multiple objects 230A-C and scores the selection using the scale 205. In the second strategy, an object 270 in the AR environment is blurred by the application by displaying a mask overlay on top of the object. The user can guess the object 270 and provide a response using the microphone of the AR headset. If the user is unsure, the user can select the option 255 to reduce the blurring of the object 270 or the option 257 to increase the intensity of the aroma.

In some implementations, the application after receiving the user response, can transmit the user response to the OTS of the server 130. Prior to transmitting the user response to the OTS, the application can analyze the user response and transmit either the user selection or the final result of the user selection. For example, if the user provided the response using the microphone of the AR headset, the application can convert audio data of the microphone to textual data. In another example, the application can analyze the user response to determine whether the user selection of the object was correct. In such a situation the application can transmit a binary value to the OTS where “1” can indicate that the user's response was correct and “0” can indicate that the user's response was wrong. Note that this reduces network traffic and reduces computational load on the server 130.

In some implementations, the OTS after receiving the user response can update the user status data based on the object of the olfaction test undertaken by the user, the aroma of the object and the user response. For example, the OTS can create a new entry in the historical data indicating the object in the AR environment that was used for the olfaction test and the aroma of the object including the intensity of the aroma detected by the aroma sensor 126 and that the user correctly identified the object and vice-versa and the score provided by the user.

In some implementations, the OTS after receiving the user response, can determine another object (referred to as a second object) that should be presented to the user in the same training session. To determine the next object, the OTS can again process historical data using the one or more machine learning models. Upon determination of the second object, the OTS can provide instructions for highlighting the second object and instructions for releasing dispersing agent that has an aroma (referred to as a second aroma) like the second object if necessary based on the intensity of aroma as detected by the aroma sensor 126.

In some implementations, the olfaction tests may require the user to move in the AR environment. This allows the OTS to generate a more immersive olfaction test in AR environment that the user can enjoy. For example, the OTS can create an olfaction test by detecting multiple objects in the three dimensional room in the AR environment. In such a scenario, the aroma sensor 126 of the AR headset can detect different aromas in the vicinity of the user while the user moves in the three dimensional room. As the user moves closer a particular object among the multiple objects that is the source of the aroma, AR headset can update the OTS about the user's co-ordinates inside the three dimensional room. Depending upon how close the user is to the particular object, the OTS can transmit instructions to alter the intensity of the release of the dispersing agent if required. Note that the AR headset will release aroma when the OTS determines that the aroma intensity of the particular object is not sufficient for the user to undertake the olfaction test.

Continuing with the above example, the user of the AR headset continues to move inside the three dimensional room. When the user selects an object from among the multiple objects in the room, the user selection is recorded as a response that transmitted to the OTS. The OTS after receiving the user response, evaluates the response to determine whether the user selection is correct. The OTS can then transmit further instructions to the AR headset to notify the user about whether the selected object is the particular object. If the selected object is not the particular object, the user can continue with the test.

Using such methods the OTS can also create a training session as a story so as to make the olfaction test more engaging to the user. For example, the training session can include the three dimensional room to be depicted as a crime scene. The user can move within the room and where the user can play a role of a detective. In this example, the user can uncover secret hidden in the room by undertaking different olfaction tests presented to the user as a part of the story. For example, as the user moves inside the three dimensional room the OTS can transmit instructions to identify different objects based on the aroma identified by the aroma sensor 126.

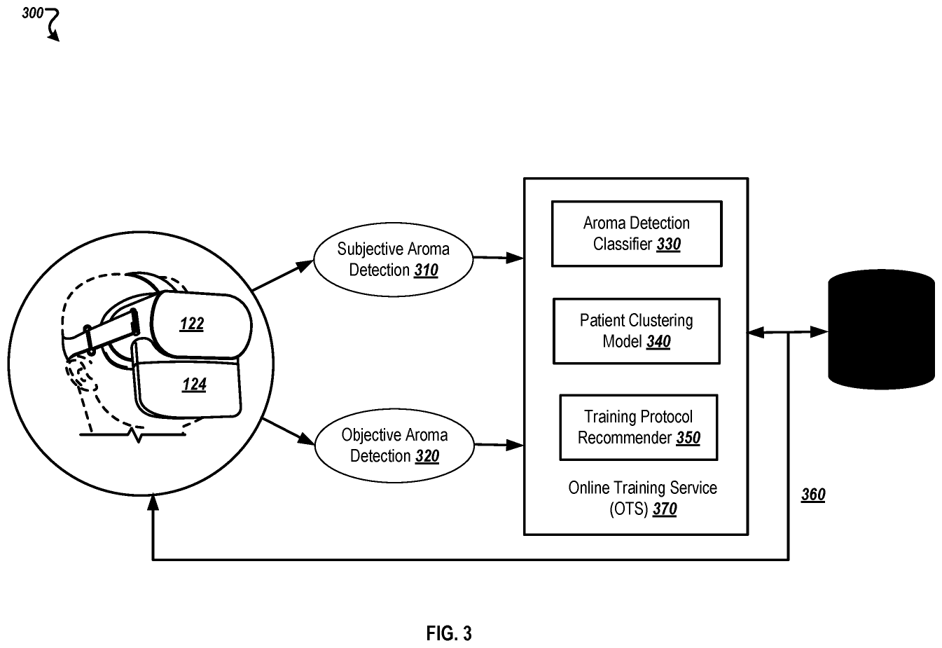

FIG. 3 is a block diagram illustrating an example OTS 370 that utilizes brain activity of the user of the AR headset. Operations explained with reference to FIG. 3 can be implemented, for example, by the AR headset and one or more servers executing the OTS 370. Operations can be implemented as instructions stored on one or more computer readable media which may be non-transitory, and execution of the instructions by one or more data processing apparatus can cause the one or more data processing apparatus to perform the operations.

As seen in FIG. 3, the AR headset 120 receives instructions 360 from the OTS 370. The instructions 360 includes data of the AR environment presented by the display 122 of the AR headset. For example, the data can include identification of an object in the user's physical environment that can be highlighted. The instructions can also include instructions to disperse aroma by the aroma dispenser 124 if necessary. After highlighting an object in the AR environment, the user tries to identify the aroma. For example, the user of the AR headset can detect aroma using the sense of olfaction (referred to as subjective aroma detection 310). It is also possible that the user of the AR headset is unable to detect the dispersed aroma however AR headset is able to detect fluctuations in electrical activity of the user's brain using EEG methods (referred to as objective aroma detection 320).

After detecting the aroma of the highlighted object, the indication of whether the aroma was detected by the user of the AR headset is transmitted to the OTS 370. For example, in case of subjective aroma detection 310, the user of the AR headset can provide an indication by interacting with the AR environment. In case of objective aroma detection, the AR headset can either process the user's brain signals to generate an indication of whether the user actually detected any aroma or transmit the brain signals to the OTS 370. In the latter case, the OTS 370 can process the brain signals to generate the indication of whether the user actually detected any aroma.

In some implementations, the OTS 370 can implement an aroma detection classifier 330 that is trained process the indications of whether the aroma was detected by the user of the AR headset and classify whether the user was actually able to detect any aroma. For example, the user may subjectively fail to detect the dispersed aroma but objectively be able to detect the aroma. After classifying the user, the OTS 370 can use a patient clustering model 340 to classify the patient into one or the multiple categories of users enrolled with the OTS 370. For example, users that were objectively able to detect aroma but unable to detect aroma subjectively can be classified as a group of users with similar characteristics. The users can further be classified in sub-categories based on user profile.

In some implementations, the OTS 370 can use a training protocol recommender 350 to process user profile of the user of the AR headset along with the outputs of the aroma detection classifier 330 and the patient clustering model 340 to generate a recommendation for an object in the physical environment of the user for a subsequent olfaction test. The recommended olfaction test that includes instructions for highlighting the recommended object in the AR environment is transmitted back to the AR headset of the user. In some implementations, the outputs of the aroma detection classifier 330, the patient clustering model 340 along with the indications of aroma detection (including subjective and objective aroma detection) is stored in the profile database 152.

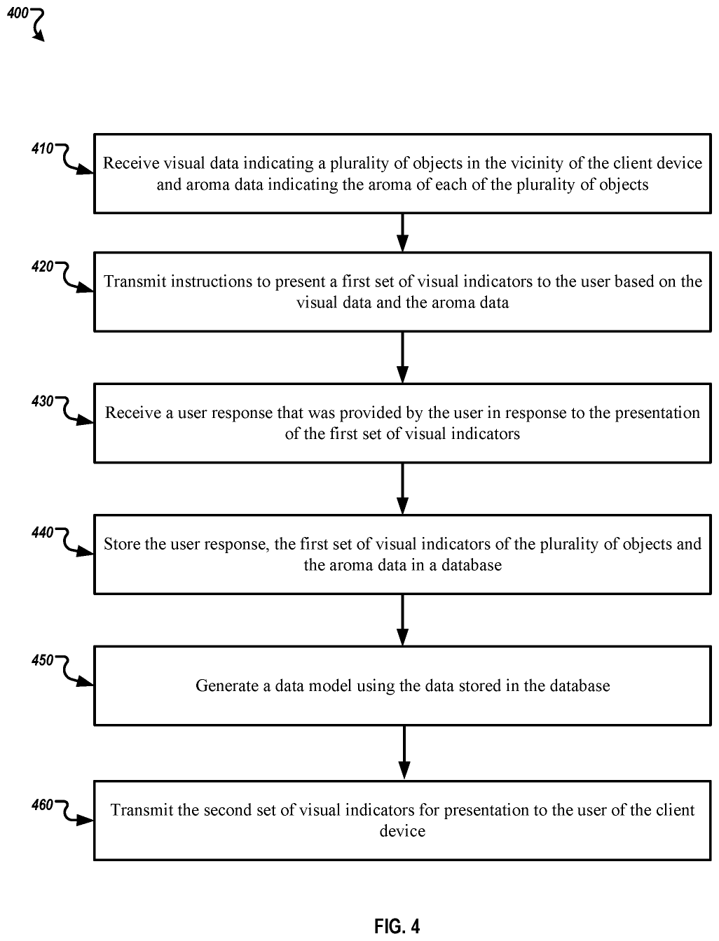

FIG. 4 is a flow diagram of an example process 400 of the olfaction training program. Operations of process 400 are described below as being performed by the components of the system described and depicted in FIGS. 1-3. Operations of the process 400 are described below for illustration purposes only. Operations of the process 400 can be performed by any appropriate device or system, e.g., any appropriate data processing apparatus. Operations of the process 400 can also be implemented as instructions stored on a non-transitory computer readable medium. Execution of the instructions cause one or more data processing apparatus to perform operations of the process 400.

Receive visual data indicating a plurality of objects in the vicinity of the client device and aroma data indicating the aroma of each of the plurality of objects (410). For example, if the user of the AR headset walks into a kitchen, the camera 128 of the AR headset can record the real-world environment of the kitchen. The real-world environment of the kitchen can include multiple objects such as fruits, bin or spices. Simultaneously, the aroma sensor 126 can also detect different aromas present in the kitchen. For example, the multiple objects such as fruits, bin or spices can have aroma that can be detected. The AR headset 120 then transmits the recorded visual data and aroma data to the one or more servers that provide an online OD testing service.

Transmit instructions to present a first set of visual indicators to the user based on the visual data and the aroma data (420). The OD testing service can process the recorded visual data and aroma data to identify one or more objects present in the vicinity of the user. The OTS in response to receiving and processing the visual data and aroma data, transmits a first set of visual instructions to the AR headset that when executed on the AR headset, highlights the identified object while presenting the real-world environment. For example, the AR headset can highlight the “orange” if the OTS decides to test if the user of the AR headset is able to perceive the aroma of the orange. In some implementations, if the user is not able to perceive the aroma, the AR headset can increase the intensity of the aroma by dispersing aroma using the aroma dispenser 124.

Receive a user response that was provided by the user in response to the presentation of the first set of visual indicators (430). For example, the user of the AR headset can interact with the AR environment to indicate whether the user is able to perceive an aroma of the highlighted object, and whether the user is able to conclude that the aroma is related to the object highlighted in the AR environment.

Store the user response, the first set of visual indicators of the plurality of objects and the aroma data in a database (440). The OTS can generate a profile for the user and store it in a database 150. User profiles can also include user status data that can incorporates information such as log data indicating the training sessions that the user has undertaken. Log data can include information such as the software and hardware configuration of the AR headset including the application that is being executed in the AR headset, the geographical location of the AR headset and the quality of the network to which the AR headset is connected to. User status data can also include historical data indicating different objects and the associated aromas that the user came across in the AR environment. Historical data can also include (or point to) the digital content that was presented to the user, the object of the AR environment, data identifying the associated aroma of the object, the intensity of the aroma of the object (recorded using the aroma sensor) and whether or not the OTS dispersed aroma to increase the intensity of the aroma. User status data can also include user responses that were recorded by the AR headset and transmitted to the OTS. The user responses can sometimes be for example, user interactions that indicate can outcome of a positive or a negative identification of an aroma of an object depicted in the AR environment, a score that indicates an association between the object and the aroma or brain activity recorded by the electrodes of the AR headset.

Generate a data model using the data stored in the database (450). For example, the OTS can access the user profile to analyze historical records of the user and the status of the user's training program to determine an object for a subsequent olfaction test. For this the OTS can implement one or more rule-based models or machine learning models. The OTS can use the rule-based models and the machine learning models to determine based on the historical records, the previous treatment sessions undertaken by the user and how well the user performed in the olfaction tests in the previous sessions.

The machine learning models implemented by the OTS can include training parameters that can be trained on historical data to predict objects that when presented to the user will improve user's OD by progressively making the user identify different aromas that the user was previously familiar with. The machine learning models implemented by the OTS are configured to receive as input information from the user profile, user status data and historical data. For example, one of the machine learning models can be a neural network model that includes multiple neural network layers and each neural network layer can include multiple training parameters. The neural network can receive as input information from user profile include for example, age, gender and the date from when the user started showing symptoms of OD. The neural network can also receive as input information from one or more previous olfaction tests. For example, the object that the user of the AR headset comes across, the user response etc. The neural network can also be configured to receive user group characteristics as input. The neural network model can process the input and generate as output a prediction indicating one or more objects that are present in the user's vicinity that can be highlighted for the user in the forthcoming olfaction test. Though the above example has been explained using a neural network model, the OTS can implement any machine learning model known in the art. For example, clustering, support vector machines (SVM).

Transmit the second set of visual indicators for presentation to the user of the client device (460). The model an process the input and generate as output a prediction indicating one or more objects that are present in the user's vicinity that can be highlighted for the user in the subsequent olfaction test. Upon determination of the one or more object that can be highlighted to the user for a subsequent olfaction test, the OTS can transmit instructions to present a set of visual indicators to the user based on the visual data highlighting the object and instructions for releasing dispersing agent if necessary. In some implementations, the AR headset after receiving the instructions from the OTS, highlights the object in the AR environment of the user of the AR headset

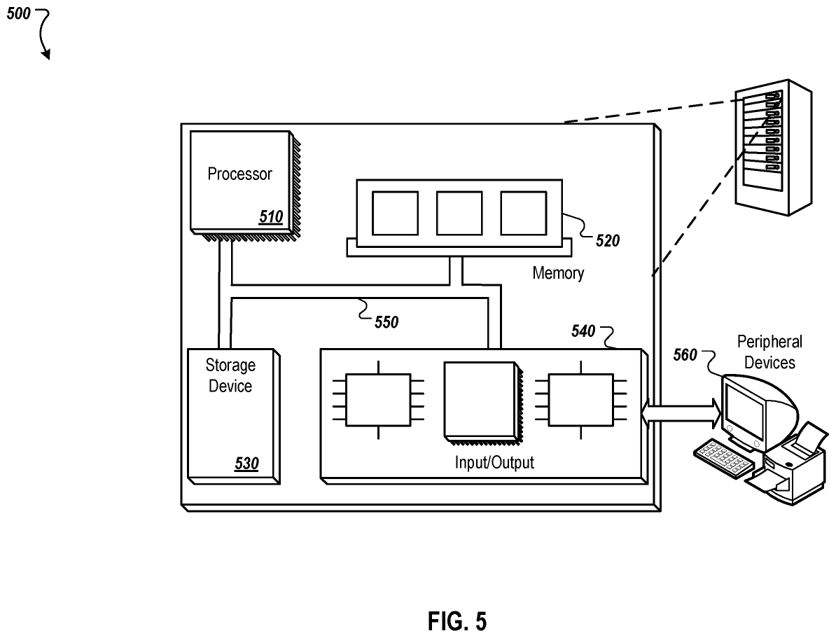

FIG. 5 is a block diagram of an example computer system 500 that can be used to perform operations described above. The system 500 includes a processor 510, a memory 520, a storage device 530, and an input/output device 540. Each of the components 510, 520, 530, and 540 can be interconnected, for example, using a system bus 550. The processor 510 is capable of processing instructions for execution within the system 500. In one implementation, the processor 510 is a single-threaded processor. In another implementation, the processor 510 is a multi-threaded processor. The processor 510 is capable of processing instructions stored in the memory 520 or on the storage device 530.

The memory 520 stores information within the system 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the system 400. In one implementation, the storage device 530 is a computer-readable medium. In various different implementations, the storage device 530 can include, for example, a hard disk device, an optical disk device, a storage device that is shared over a network by multiple computing devices (e.g., a cloud storage device), or some other large capacity storage device.

The input/output device 540 provides input/output operations for the system 500. In one implementation, the input/output device 540 can include one or more of a network interface devices, e.g., an Ethernet card, a serial communication device, e.g., and RS-232 port, and/or a wireless interface device, e.g., and 802.11 card. In another implementation, the input/output device can include driver devices configured to receive input data and send output data to peripheral devices 560, e.g., keyboard, printer and display devices. Other implementations, however, can also be used, such as mobile computing devices, mobile communication devices, set-top box television client devices, etc.

Although an example processing system has been described in FIG. 5, implementations of the subject matter and the functional operations described in this specification can be implemented in other types of digital electronic circuitry, or in computer software, firmware, or hardware, including the structures disclosed in this specification and their structural equivalents, or in combinations of one or more of them.

An electronic document (which for brevity will simply be referred to as a document) does not necessarily correspond to a file. A document may be stored in a portion of a file that holds other documents, in a single file dedicated to the document in question, or in multiple coordinated files.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage media (or medium) for execution by, or to control the operation of, data processing apparatus. Alternatively, or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a sub combination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A computer-implemented method of an olfaction training program, comprising:
receiving from a client device of an user, visual data indicating a plurality of objects in the vicinity of the client device and aroma data indicating the aroma of each of the plurality of objects;
transmitting to the client device, instructions to present a first set of visual indicators to the user based on the visual data and the aroma data;
receiving from the client device, a user response that was provided by the user in response to the presentation of the first set of visual indicators;
storing data in a database wherein the data includes the user response, the first set of visual indicators of the plurality of objects and the aroma data;
generating a data model using the data stored in the database wherein the data model is configured to generate as output, a second set of visual indicators for presentation to the user of the client device;
transmitting, to the client device, the second set of visual indicators for presentation to the user of the client device.

2. The computer-implemented method of claim 1, wherein visual data comprises data indicating the plurality of objects in a three dimensional environment of the user of the client device collected using a camera of the client device.

3. The computer-implemented method of claim 1, wherein aroma data comprises data indicating the aroma of one or more objects in the plurality of objects collected using an aroma sensor of the client device.

4. The computer-implemented method of claim 1, wherein the first set of visual indicators comprises (i) visual data identifying one or more objects from among the plurality of objects, (ii) visual data depicting the intensity of aroma of the one or more objects.

5. The computer-implemented method of claim 1, wherein instructions to highlight the first set of visual indicators to identify the one or more objects from among the plurality of objects.

6. The computer-implemented method of claim 5, wherein the user response provided by the user in response to the presentation of the first set of visual indicators comprises (1) an indication whether the user was able to perceive the aroma of the one or more objects, (2) an indication whether the user is able to associate the aroma of the one or more objects with the one or more objects displayed on the client device, and (3) a score provided by the user indicating the level of confidence the user has on the association of the one or more aromas of the one or more objects to the one or more objects present by the client device.

7. The computer-implemented method of claim 6, wherein the user response provided by the user can further include electrical activity of the brain of the user collected using one or more electrodes of the client device affixed to the scalp of the user wherein the electrical activity of the brain identifies brain activity in response to the user smelling the first object.

8. The computer-implemented method of claim 1, wherein generating the data model comprises:
identifying one or more users from among the plurality of users based on prior user responses;
generating a training dataset based on data stored in the database of the one or more users;
generating the data model based on the training dataset that further comprises:
generating a rule-based data model that includes a set of rules that generates as output, the second data that indicates the second object and the second smell;
generating a machine learning data model that is trained on the one or more identified training datasets to generate as output, the second data that indicates the second object and the second smell.

9. A system, comprising:
receiving from a client device of an user, visual data indicating a plurality of objects in the vicinity of the client device and aroma data indicating the aroma of each of the plurality of objects;
transmitting to the client device, instructions to present a first set of visual indicators to the user based on the visual data and the aroma data;
receiving from the client device, a user response that was provided by the user in response to the presentation of the first set of visual indicators;
storing data in a database wherein the data includes the user response, the first set of visual indicators of the plurality of objects and the aroma data;
generating a data model using the data stored in the database wherein the data model is configured to generate as output, a second set of visual indicators for presentation to the user of the client device;
transmitting, to the client device, the second set of visual indicators for presentation to the user of the client device.

10. The system of claim 9, wherein visual data comprises data indicating the plurality of objects in a three dimensional environment of the user of the client device collected using a camera of the client device.

11. The system of claim 9, wherein aroma data comprises data indicating the aroma of one or more objects in the plurality of objects collected using an aroma sensor of the client device.

12. The system of claim 9, wherein the first set of visual indicators comprises (i) visual data identifying one or more objects from among the plurality of objects, (ii) visual data depicting the intensity of aroma of the one or more objects.

13. The system of claim 9, wherein instructions to highlight the first set of visual indicators to identify the one or more objects from among the plurality of objects.

14. The system of claim 13, wherein the user response provided by the user in response to the presentation of the first set of visual indicators comprises (1) an indication whether the user was able to perceive the aroma of the one or more objects, (2) an indication whether the user is able to associate the aroma of the one or more objects with the one or more objects displayed on the client device, and (3) a score provided by the user indicating the level of confidence the user has on the association of the one or more aromas of the one or more objects to the one or more objects present by the client device.

15. The system of claim 14, wherein the user response provided by the user can further include electrical activity of the brain of the user collected using one or more electrodes of the client device affixed to the scalp of the user wherein the electrical activity of the brain identifies brain activity in response to the user smelling the first object.

16. The system of claim 1, wherein generating the data model comprises:
identifying one or more users from among the plurality of users based on prior user responses;
generating a training dataset based on data stored in the database of the one or more users;
generating the data model based on the training dataset that further comprises:
generating a rule-based data model that includes a set of rules that generates as output, the second data that indicates the second object and the second smell;
generating a machine learning data model that is trained on the one or more identified training datasets to generate as output, the second data that indicates the second object and the second smell.

17. A non-transitory computer readable medium storing instructions that, when executed by one or more data processing apparatus, cause the one or more data processing apparatus to perform operations comprising:
receiving from a client device of an user, visual data indicating a plurality of objects in the vicinity of the client device and aroma data indicating the aroma of each of the plurality of objects;
transmitting to the client device, instructions to present a first set of visual indicators to the user based on the visual data and the aroma data;
receiving from the client device, a user response that was provided by the user in response to the presentation of the first set of visual indicators;
storing data in a database wherein the data includes the user response, the first set of visual indicators of the plurality of objects and the aroma data;
generating a data model using the data stored in the database wherein the data model is configured to generate as output, a second set of visual indicators for presentation to the user of the client device;
transmitting, to the client device, the second set of visual indicators for presentation to the user of the client device.

18. The non-transitory computer readable medium of claim 17, wherein visual data comprises data indicating the plurality of objects in a three dimensional environment of the user of the client device collected using a camera of the client device.

19. The non-transitory computer readable medium claim 17, wherein aroma data comprises data indicating the aroma of one or more objects in the plurality of objects collected using an aroma sensor of the client device.

20. The non-transitory computer readable medium of claim 17, wherein the first set of visual indicators comprises (i) visual data identifying one or more objects from among the plurality of objects, (ii) visual data depicting the intensity of aroma of the one or more objects.
